(19) United States
(12) Patent Application Publication
(10) Pub. No.: US 2008/0077180 A1
(43) Pub. Date: Mar. 27, 2008

(54) Scaffold for tubular septal occluder device and techniques for attachment

(75) Inventors: Stephanie M. Kladakis, Stoneham, MA (US); David P. Marchesiello, Boston, MA (US); Carol A. Devellian, Topsfield, MA (US)

Correspondence Address: WILMERHALE/BOSTON, 60 STATE STREET, BOSTON, MA, 02109, US

(73) Assignee: NMT Medical, Inc., Boston, MA (US)

(21) Appl. No.: 11/904,137

(22) Filed: Sep. 26, 2007

Related U.S. Application Data
(60) Provisional application No. 60/847,352, filed on Sep. 26, 2006.

(51) Int. Cl. A61B 17/08

(52) U.S. Cl. 606/216

(57) ABSTRACT

The present invention provides a device for occluding an anatomical aperture, such as an atrial septal defect (ASD) or a patent foramen ovale (PFO). The occluder includes two sides connected by a central tube. A tissue scaffold material is disposed on the occluder. The occluder is formed from a tube, which is cut to produce struts in each side. Upon the application of force, the struts deform into loops. The loops may be of various shapes, sizes, and configurations, and, in at least some embodiments, the loops have rounded peripheries. In some embodiments, at least one side of the occluder includes a tissue scaffold. The occluder further includes a catch system that maintains its deployed state in vivo. When the occluder is deployed in vivo, the two sides are disposed on opposite sides of the septal tissue surrounding the aperture and the catch system is deployed so that the occluder exerts a compressive force on the septal tissue and closes the aperture.

### CROSS REFERENCE TO RELATED APPLICATIONS

This application claims the benefit of provisional application U.S. Ser. No. 60/847,352 filed Sep. 26, 2006, the entire contents of which is incorporated by reference.

### FIELD OF THE INVENTION

The present invention relates generally to an occlusion device for the closure of physical anomalies, such as an atrial septal defect, a patent foramen ovale, and other septal and vascular defects.

### BACKGROUND OF THE INVENTION

A patent foramen ovale (PFO), illustrated in FIG. 1, is a persistent, one-way, usually flap-like opening in the wall between the right atrium 11 and left atrium 13 of the heart 10. Because left atrial (LA) pressure is normally higher than right atrial (RA) pressure, the flap usually stays closed. Under certain conditions, however, right atrial pressure can exceed left atrial pressure, creating the possibility that blood could pass from the right atrium 11 to the left atrium 13 and blood clots could enter the systemic circulation. It is desirable that this circumstance be eliminated.

The foramen ovale serves a desired purpose when a fetus is gestating in utero. Because blood is oxygenated through the umbilical chord, and not through the developing lungs, the circulatory system of the fetal heart allows the blood to flow through the foramen ovale as a physiologic conduit for right-to-left shunting. After birth, with the establishment of pulmonary circulation, the increased left atrial blood flow and pressure results in functional closure of the foramen ovale. This functional closure is subsequently followed by anatomical closure of the two over-lapping layers of tissue: septum primum 14 and septum secundum 16. However, a PFO has been shown to persist in a number of adults.

The presence of a PFO is generally considered to have no therapeutic consequence in otherwise healthy adults. Paradoxical embolism via a PFO is considered in the diagnosis for patients who have suffered a stroke or transient ischemic attack (TIA) in the presence of a PFO and without another identified cause of ischemic stroke. While there is currently no definitive proof of a cause-effect relationship, many studies have confirmed a strong association between the presence of a PFO and the risk for paradoxical embolism or stroke. In addition, there is significant evidence that patients with a PFO who have had a cerebral vascular event are at increased risk for future, recurrent cerebrovascular events.

Accordingly, patients at such an increased risk are considered for prophylactic medical therapy to reduce the risk of a recurrent embolic event. These patients are commonly treated with oral anticoagulants, which potentially have adverse side effects, such as hemorrhaging, hematoma, and interactions with a variety of other drugs. The use of these drugs can alter a person's recovery and necessitate adjustments in a person's daily living pattern.

In certain cases, such as when anticoagulation is contraindicated, surgery may be necessary or desirable to close a PFO. The surgery would typically include suturing a PFO closed by attaching septum secundum to septum primum. This sutured attachment can be accomplished using either an interrupted or a continuous stitch and is a common way a surgeon shuts a PFO under direct visualization.

Umbrella devices and a variety of other similar mechanical closure devices, developed initially for percutaneous closure of atrial septal defects (ASDs), have been used in some instances to close PFOs. These devices potentially allow patients to avoid the side effects often associated with anticoagulation therapies and the risks of invasive surgery. However, umbrella devices and the like that are designed for ASDs are not optimally suited for use as PFO closure devices.

Currently available septal closure devices present drawbacks, including technically complex implantation procedures. Additionally, there are not insignificant complications due to thrombus, fractures of the components, conduction system disturbances, perforations of heart tissue, and residual leaks. Many devices have high septal profile and include large masses of foreign material, which may lead to unfavorable body adaptation of a device. Given that ASD devices are designed to occlude holes, many lack anatomic conformability to the flap-like anatomy of PFOs. Thus, when inserting an ASD device to close a PFO, the narrow opening and the thin flap may form impediments to proper deployment. Even if an occlusive seal is formed, the device may be deployed in the heart on an angle, leaving some components insecurely seated against the septum and, thereby, risking thrombus formation due to hemodynamic disturbances. Finally, some septal closure devices are complex to manufacture, which may result in inconsistent product performance.

The present invention is designed to address these and other deficiencies of prior art septal closure devices.

### SUMMARY OF THE INVENTION

In one aspect, the present invention provides a device for occluding an aperture in septal tissue, including a first side adapted to be disposed on one side of the septal tissue and a second side adapted to be disposed on the opposite side of the septal tissue. The first and second sides are adapted to occlude the aperture upon deployment of the device at its intended delivery location. The device also includes a catch system that maintains the configuration of the device once it has been deployed.

According to some embodiments, the catch system reduces and maintains the axial length of the device. Also, varied constructions could be used to maintain the axial dimension of the device. In one form, catch elements such as, e.g., balls, attached to a delivery wire could be used to maintain the axial dimension of the device. In a different construction, a locking mechanism could be used. Preferably, if a locking mechanism is used, it secures both sides of the device in the locked position with a single locking element.

According to at least some embodiments, the device is formed from a tube. According to some embodiments, the tube includes a material selected from the group consisting of metals, shape memory materials, alloys, polymers, bioabsorbable polymers, and combinations thereof. In particular embodiments, the tube includes a shape memory polymer. According to some embodiments, the device is formed by cutting the tube.

According to some embodiments of the present invention, at least one of the first and second sides of the device includes a tissue scaffold. According to some embodiments, the tissue scaffold includes a material selected from the group consisting of polyester fabrics, Teflon-based materials, polyurethanes, metals, polyvinyl alcohol (PVA), extracellular matrix (ECM), purified bioengineered type I collagen, derived from a tunica submucosa layer of a porcine small intestine or other bioengineered materials, synthetic bioabsorbable polymeric scaffolds, collagen, and combinations thereof. In particular embodiments, the tissue scaffold includes nitinol. The tissue scaffold may completely or partially encase the occluder and, in particular, the proximal and distal petals. The tissue scaffold may be constructed by piecing together precut, shaped components that when assembled closely approximate the three-dimensional shape of the occluder. Different embodiments incorporate different seam patterns that offer different edge profiles, which can determine what type and size of sheath is most suitable for a particular occluder. The tissue scaffold can be disc shaped and attached to one or more sides of the loops or arms of the occluder. Additionally, the tissue scaffold may have a shaped contour at its outer edge, which can conform to the outline of the occluder. Further, the contour may also be oriented to extent radially outward between the loops. A method of assembling the scaffold is also disclosed.

According to some embodiments, the first and second sides of the device are connected by a central tube. According to some embodiments, the central tube is positioned so as to minimize distortion to the septal tissue surrounding the aperture. In particular embodiments, the central tube is positioned at an angle θ from the second side, and the angle θ is greater than 0 degrees and less than about 90 degrees.

In another aspect, embodiments of the invention provide a device for occluding an aperture in septal tissue, including a first side adapted to be disposed on one side of the septal tissue and a second side adapted to be disposed on the opposite side of the septal tissue. The first and second sides are adapted to occlude the defect when the device is deployed at its intended delivery location. Each of the first and second sides includes loops. The device further includes a catch system that maintains the configuration of the device once it has been deployed. The loops of the first and second sides and the catch system cooperate to provide a compressive force to the septal tissue surrounding the aperture.

According to some embodiments, each of the first and second sides includes at least two loops. In particular embodiments, each of the first and second sides includes four or six loops. Of course, the most desirable number of loops on each side will depend on a variety of anatomical and manufacturing factors.

According to some embodiments, each of the loops includes a rounded edge at its periphery to minimize trauma to the septal tissue. In particular embodiments, the outer periphery of the device is circular.

In still another aspect, embodiments of the invention provide a method of making a device for occluding an aperture in septal tissue, including providing a tube having first and second ends and upper and lower portions, cutting at least four axially-extending openings in the upper portion of the tube, cutting at least four axially-extending openings in the lower portion of the tube. The openings in the upper and lower portions are separated by a central portion of the tube.

According to some embodiments, the tube includes a material selected from the group consisting of metals, shape memory materials, alloys, polymers, bioabsorbable polymers, and combinations thereof. In particular embodiments, the tube includes a shape memory polymer.

In yet another aspect, some described embodiments provide a method of occluding an aperture in septal tissue, including providing a tube having first and second ends and upper and lower portions in a delivery sheath. The tube includes at least four axially-extending openings in its upper portion and at least three axially-extending openings in its lower portion. The openings in the upper and lower portions are separated by a central portion of the tube. The deliver sheath is inserted into a right atrium of a heart, through the aperture in the septal tissue, and into the left atrium of the heart. The first end and the upper portion of the tube are deployed into the left atrium. The sheath is then retracted through the aperture and into the right atrium of the heart, where the second end and the lower portion of the tube are deployed into the right atrium. The sheath is then withdrawn from the heart. Of course, a catch system could be used to secure the device in a delivered (expanded) state. The catch system may have any or all the characteristics described in the specification. Further, other types of catch systems could be used to hold the device in the delivered state.

According to some embodiments, a force is applied to each of the first and second ends in an axial direction such that the axial length of the tube is reduced. The force applied to the first end is in a direction opposite to that of the force applied to the second end.

### BRIEF DESCRIPTION OF THE DRAWINGS

FIG. 1 is a schematic representation of a human heart including various septal defects;

FIGS. 2A-2D are isometric views of an embodiment of an occluder according to the present invention;

FIGS. 2E-2H are isometric views of an embodiment of an occluder according to the present invention;

FIGS. 2I-2K are isometric views of occluders according to various embodiments of the invention;

FIGS. 2L and 2M are side and top views, respectively, of an alternate embodiment of an occluder according to the present invention;

FIGS. 2N, 2P, 2Q, and 2R are end views of various embodiments of a tubular occluder (FIG. 2′O′ is not used);

FIGS. 3A-3C are front elevational, side, and cross-sectional views, respectively, of the occluder of FIGS. 2A-2D;

FIGS. 4A-4B are front elevational and side views, respectively, of another embodiment of an occluder according to the present invention;

FIGS. 5A-5B are front and side views, respectively, of still another embodiment of an occluder according to the present invention;

FIGS. 6A-6C are isometric views of one embodiment of a catch system according to the present invention;

FIGS. 7A-7B are side views of one method for delivering an occluder according to the present invention to a septal defect;

FIG. 8 is a side view of the occluder of FIGS. 2I-2K deployed in vivo;

FIG. 9 is a perspective view of one embodiment of an occluder with a tissue scaffold according to the present invention;

FIG. 10 is a cut-out pattern for a portion of the tissue scaffold illustrated in FIG. 9 according to one embodiment of the present invention;

FIG. 11 is a perspective view of one embodiment of an occluder with a tissue scaffold according to the present invention;

FIG. 12 is a cut-out pattern for a portion of the tissue scaffold illustrated in FIG. 11, according to one embodiment of the present invention;

FIG. 13 is a perspective view of one embodiment of an occluder with a tissue scaffold, according to the present invention;

FIGS. 14A-C illustrate alternative cut-out patterns for various embodiments of the tissue scaffold illustrated in FIG. 13, according to the present invention;

FIG. 15 is a schematic representation of an occluder according to the present invention identifying the locations for possible attachment of a tissue scaffold;

FIG. 16 is a schematic representation of an occluder with a tissue scaffold partially attached;

FIGS. 17 and 18 are schematic representations of an end view of an occluder with an alternate embodiment of the tissue scaffold; and,

FIG. 19 is a schematic representation of an occluder with tissue scaffolding illustrating a possible size combination.

### DETAILED DESCRIPTION OF THE INVENTION

The present invention provides a device for occluding an aperture within body tissue. This device relates particularly to, but is not limited to, a septal occluder made from a polymer tube. In particular and as described in detail below, the occluder of the present invention may be used for closing an ASD or PFO in the atrial septum of a heart. Although the embodiments of the invention are described with reference to an ASD or PFO, one skilled in the art will recognize that the device and methods of the present invention may be used to treat other anatomical conditions. As such, the invention should not be considered limited in applicability to any particular anatomical condition.

The present invention provides a tissue scaffolding that can assist in sealing the physical condition or anomaly, e.g., PFO, by sealing the tunnel in a more complete manner than if the occluder was used alone. Also, the tissue scaffold of the present invention may be suitable for allowing tissue in-growth to assist in the process of fusing the tissue together. Further, the tissue scaffolding can be impregnated with a pharmacological material to promote some physiologic response. The tissue scaffolding of the present invention may be a one piece construction with seams that are affixed or there may be one or more pieces that are connected to cover all or part of the device. The advantages of tissue scaffolding for the implanted device, e.g., promotion of healing, should be balanced with the potential “bulkiness” which can enlarge the profile during delivery which potentially could require a larger catheter to deliver the occluder.

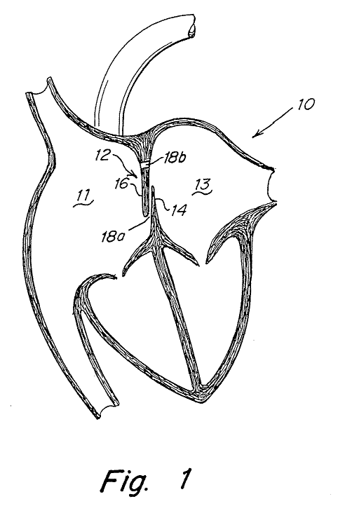

FIG. 1 illustrates a human heart 10, having a right atrium 11 and a left atrium 13 and including various anatomical anomalies 18a and 18b. The atrial septum 12 includes septum primum 14 and septum secundum 16. The anatomy of the septum 12 varies widely within the population. In some people, septum primum 14 extends to and overlaps with septum secundum 16. The septum primum 14 may be quite thin. When a PFO is present, blood could travel through the passage 18a between septum primum 14 and septum secundum 16 (referred to as “the PFO tunnel”). Additionally or alternatively, the presence of an ASD 18b could permit blood to travel through an aperture in the septal tissue, such as that schematically illustrated by aperture 18b.

The term “bioabsorbable,” as used in this application, is also understood to mean “bioresorbable.”

In this application, “distal” refers to the direction away from a catheter insertion location and “proximal” refers to the direction nearer the insertion location.

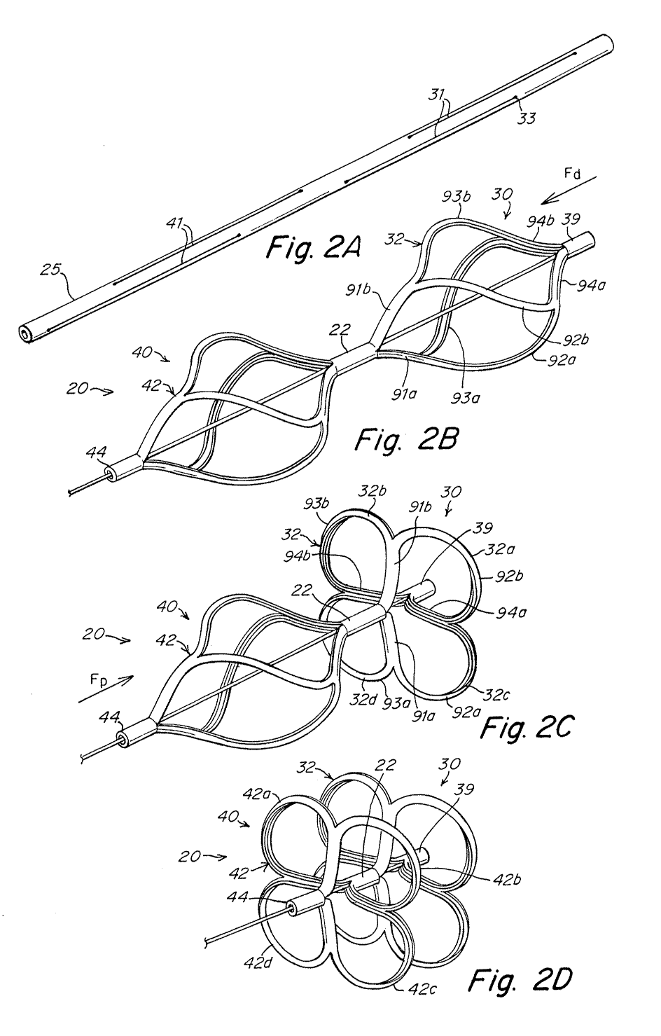

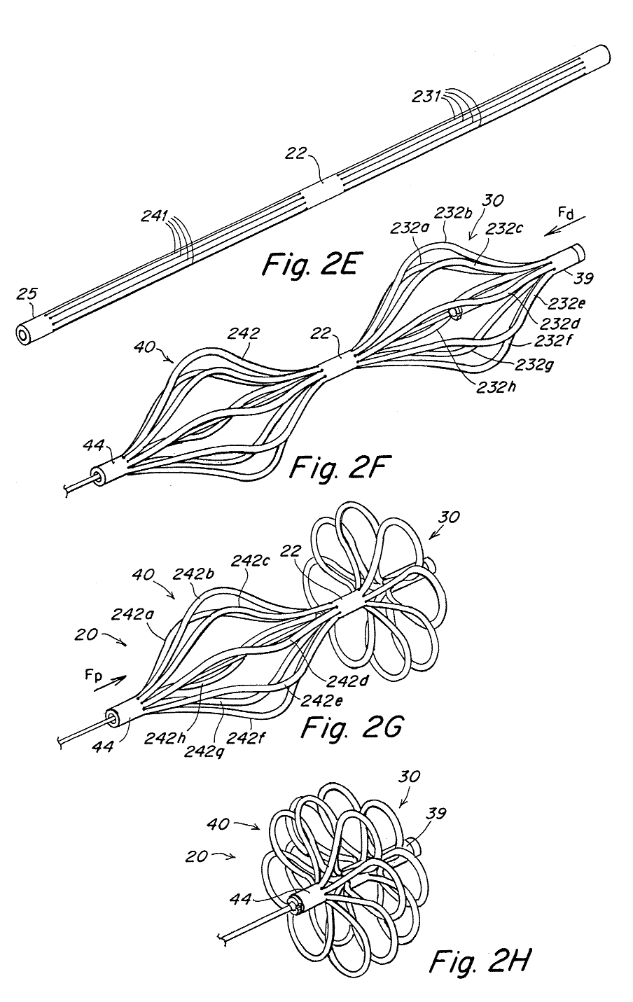

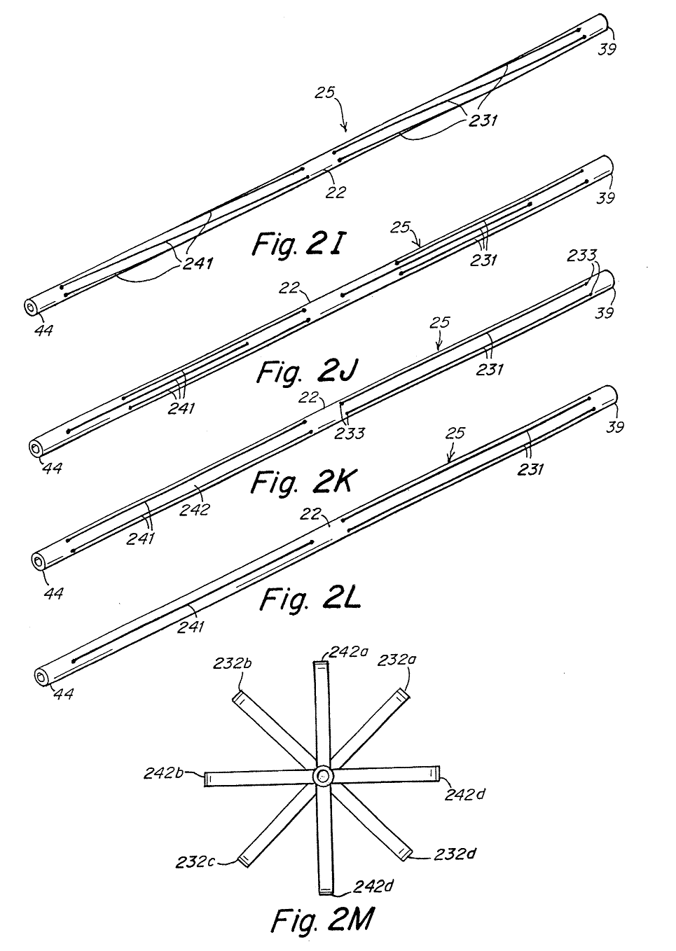

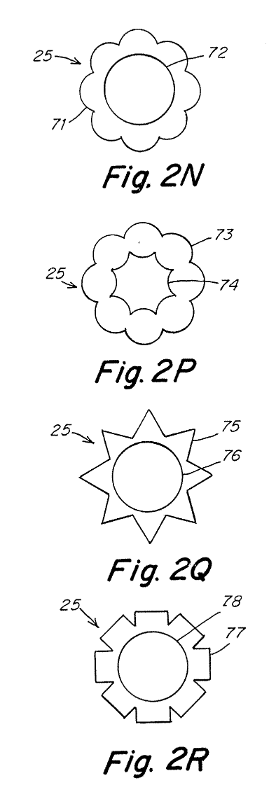

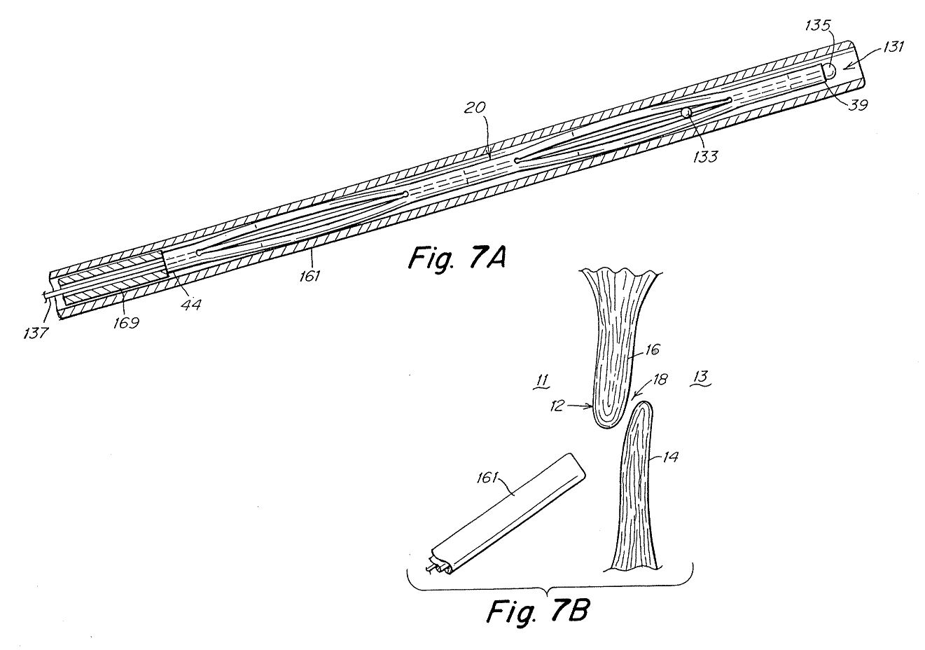

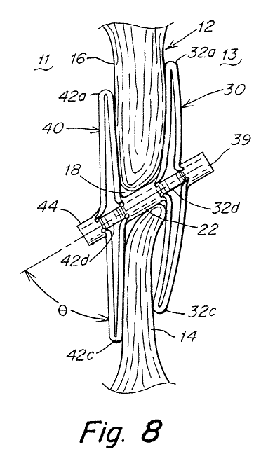

Referring to occluder 20, distal side 30 and proximal side 40 are connected by central tube 22. As illustrated, e.g., in FIGS. 2B and 2E the central tube 22 is an uncut central part of the tube used to form occluder 20. As described below, the entire tube is indicated by reference numeral 25. As shown in FIGS. 7B and 8, the occluder 20 may be inserted into the septum 12 to prevent the flow of blood through the aperture 18a, e.g., the occluder may extend through the PFO tunnel such that the distal side 30 is located in the left atrium 13 and the proximal side 40 is located in the right atrium 11. Additionally or alternatively, the occluder 20 extends through the ASD 18b so as to prevent the flow of blood through the aperture 18b such that the distal side 30 is located in the left atrium 13 and the proximal side 40 is located in the right atrium 11. As used in this application, unless otherwise indicated, the term “aperture 18” refers to any anatomical anomaly that may be treated by use of occluder 20, such as PFO 18a or ASD 18b.

The occluder 20 is constructed of one or more metal or polymer tube(s), referred to collectively as “tube” 25. Tube 25 includes slits 31 and 41 (or 231 and 241), which are formed using an etching or cutting process that produces a particular cutting pattern on tube 25. For example, as shown in FIG. 2K, slits 31 (or 231) are cut along the axial length of the upper half of tube 25 using a cutting tool, e.g., a razor blade. According to some embodiments of the present invention and as shown in FIG. 2K, slits 31 (or 231) are cut without removing any significant amount of material from tube 25, i.e., the formation of slits 31 (or 231) does not significantly reduce the overall volume of tube 25. According to other embodiments of the present invention, slits 31 (or 231) are formed by cutting material out of tube 25 such that the volume of tube 25 is reduced. Both ends of each of slits 31 (or 231) are rounded so as to relieve stresses at the axial ends of the slits 31 (or 231). This prevents slits 31 (or 231) from lengthening due to cyclic stresses present in a beating heart and the resultant material fatigue. In those embodiments where slits 31 (or 231) are cut without removing any significant amount of material from tube 25, rounded ends or holes 33 (or 233) may be produced by burning holes at both ends of each of slits 31 (or 231). In those embodiments where slits 31 (or 231) are formed by cutting material out of tube 25, rounded ends 33 (or 233) may be formed during the cutting process. The size of rounded ends 33 (or 233) may vary depending upon the dimensions of tube 25 and the amount of stress release required by the deformation.

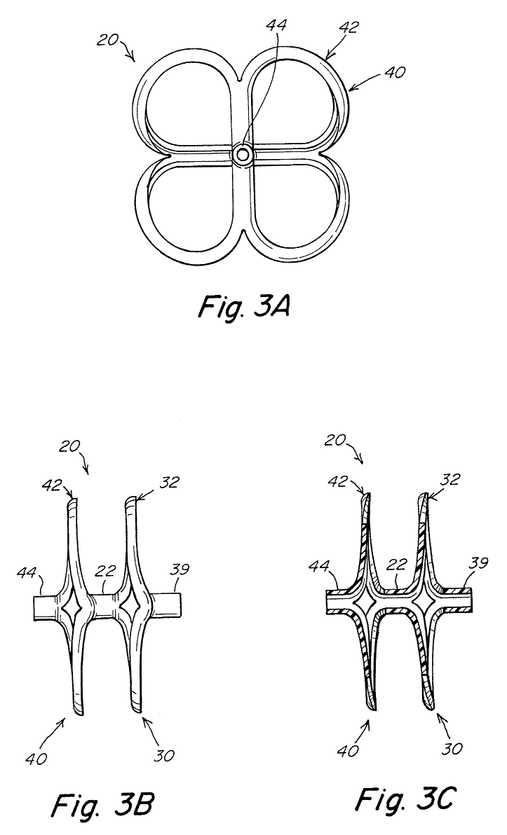

FIGS. 2D and 2H illustrate exemplary occluder 20 formed from a tube 25, according to some embodiments of the present invention. Configuration of the occluder 20 is determined by the cutting pattern on tube 25. For example, and as shown in FIGS. 2A, 2B-2D, and 3A-3C, petal-shaped loops 32, 42 (FIGS. 2A-2D and FIG. 3A) are produced by cutting slits 31 in the distal side 30 of tube 25, and cutting slits 41 in the proximal side 40 of tube 25 according to the cutting pattern shown in FIG. 2A. As shown in FIG. 2B, the distal side 30 of tube 25 is cut in half from a center portion 22 to a distal distance to form half sections 91a and 91b. The half sections 91a and 91b are further cut to a proximal distance from the distal end 39 into quarter sections 92a, 93a, 92b, and 93b. The cuts are discontinued and quarter sections 92a and 92b form half section 94a at end 39, and quarter sections 93a and 93b form half section 94b at end 39. Upon application of force F_{d} to end 39, struts bow and twist outward to form petal-shaped loops 32 in distal side 30, as shown in FIGS. 2C-2D. The movement of the struts during deployment is such that the struts rotate in an orthogonal plane relative to the axis of the device. Central tube 22 may be constrained during the application of force F_{d}, or any combination of forces sufficient to reduce the axial length of the tube 25 may be applied. One end of each of petal-shaped loops 32 originates from central tube 22, while the other end originates from end 39 (FIGS. 2B-2C and FIG. 3A). Petal-shaped loops 42 may be formed in proximal side 40 of tube 25, as shown in FIGS. 2B-2D, using the same cutting pattern described above.

According to one embodiment of the invention, the loops of the occluder are formed by struts as illustrated in FIG. 2B. Sections 91a, 91b, 92a, 92b, 93a, 93b, 94a, and 94b are of equal distance, being about ⅓ the length of distal side 30 (i.e., the distance between central tube 22 and end 39) of the tube 25. According to another embodiment of the invention, other lengths of sections can be used to produce advantageous results. In general, the longer the length of the hemispherical struts, such as half sections 91a, 91b, 94a, and 94b, the stiffer the occluder will be. The longer the length of the quarter (as shown) struts, such as half sections 92a, 92b, 93a, and 93b, the less stiff the occluder will be. In general, the hemispherical cut (one of the two) may be 20-40% of the overall length of the distal side (or proximal side) the tube. Specifically, the hemispherical cuts could be 40% of the overall length of the distal side (or proximal side) and then the quarter cut could be 20% of the overall length of the distal side (or proximal side) of the tube 25. Also, the lengths of the hemispherical cuts need not be the same. It may be advantageous to shorten one or the other side of the hemispherical cut based on a desired stiffness characteristic for a particular application of the occluder. In an alternative structure, the hemispherical cuts can be extended in a range up to 100% of the length of the distal side (or the proximal side) of the occluder, while still enabling the bow and twist of the struts.

As illustrated, the loops 32 are evenly distributed about central tube 22 and end 39. Thus, when the distal side 30 includes four loops 32 (as shown in FIGS. 2C and 2D), the four slits 31 are spaced 90 degrees radially apart. Similarly, when the distal side 30 includes six loops 32, the six slits 31 are spaced 60 degrees radially apart. The angle between radially equally-spaced is determined by the formula (360/n_{d}), where n_{d} is the total number of loops 32.

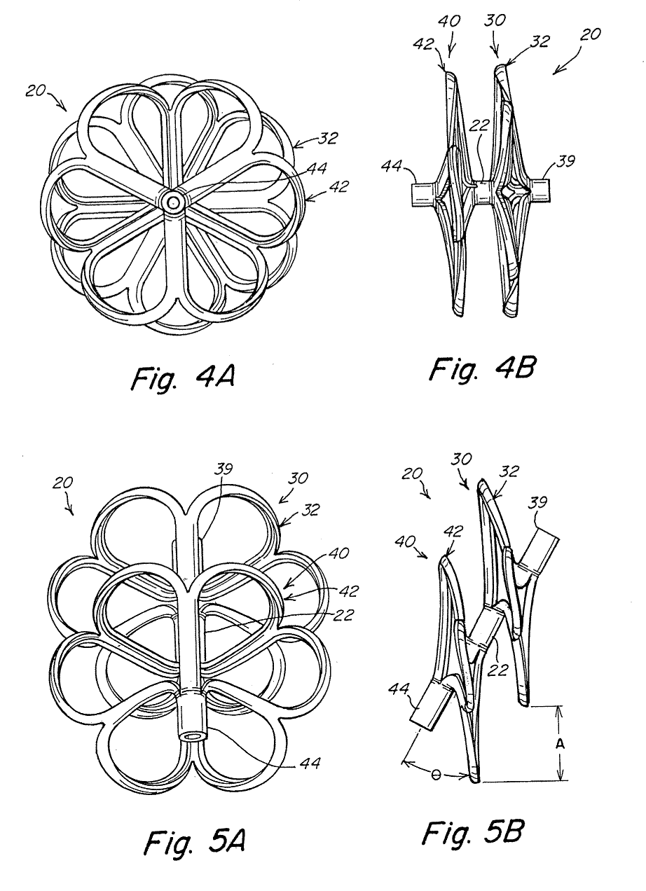

Although the distal side 30 of the occluder 20 shown in FIG. 3A includes four loops 32, occluders according to the present invention may include any number of loops 32 necessary for a given application. In particular embodiments, the distal side 30 of occluder 20 includes six loops 32 (FIG. 4A). Occluders having between four and ten loops 32 may be formed without requiring significant adjustments in the processes described in this application. However, occluders having less than four or more than ten loops 32 may be complicated to manufacture and difficult deliver through the vasculature.

The proximal side 40 of the occluder 20, shown in side view in FIG. 2D, also includes four loops, 42a, 42b, 42c, and 42d (collectively referred to as loops 42). As previously described, each of loops 42a-42d are formed by corresponding cut sections, produced by cutting slits 41. The application of force Fₚ to tip 44 of tube 25 brings the axial ends of slits 41 together such that struts bow and twist outwardly to form loops 42 of proximal side 40 (FIGS. 2C-2D). Central tube 22 may be constrained during the application of force Fₚ. One skilled in the art will recognize that any combination of forces sufficient to reduce the axial length of the tube 25 would be sufficient to deploy the proximal side 40 of occluder 20. As described above for distal loops 32, the loops 42 are evenly distributed about central tube 22 and tip 44. Similarly, the angle between radially equally-spaced slits 41 in the proximal side 40 is determined by the formula (360/n_{d}), where n_{d} is the total number of loops 42.

Although the proximal side 40 of the occluder 20 shown in FIG. 2D includes four loops 42, one skilled in the art will recognize that the proximal side 40 of an occluder according to the present invention may include any number of loops 42 required and suitable for a given application. In particular embodiments, the proximal side 40 of occluder 20 includes six loops 42 (FIG. 4A). Further, although as illustrated, distal side 30 and proximal side 40 both include four loops, there is no requirement that distal side 30 and proximal side 40 of occluder 20 include the same number of loops. In fact, in particular applications, it may be advantageous to use an occluder 20 in which the distal side 30 contains fewer loops than the proximal side 40, or vice versa.

Given that the surface of occluder 20 will contact septum 12 once it is deployed in vivo, slits 31 and 41 are cut so as to prevent the formation of sharp, potentially damaging edges along their length. For example, a heated cutting tool may be used to cut slits 31 and 41 such that the material of tube 25 melts slightly when placed in contact with the cutting tool. Such melting rounds the edges of the sections. Lasers may also be used to cut slits 31 and 41. According to this process, the edges of loops 32 and 42 formed by the cutting of slits 31 and 41 are blunted (due to melting) to prevent tissue damage in vivo. One skilled in the art will recognize that same considerations and techniques also apply to slits 31 and 41.

It will be apparent to one skilled in the art that loops 32 and loops 42 do not have to be the same size. In one embodiment, loops 32 are larger in size than loops 42. In another embodiment, loops 32 are smaller in size than loops 42. Size of loops 32 and 42 is determined by the lengths of slits 31 and 41, respectively. Therefore, absolute and relative lengths of slits 31 and 41 can be varied to achieve desired absolute and relative sizes of loops 32 and 42. Other embodiments to which the slit length may be independently varied is discussed below and further shown in FIGS. 2E-2H.

In at least some embodiments, illustrated in FIG. 4A, loops 42 of the proximal side 40 are radially offset from loops 32 of the distal side 30 to provide a better distribution of forces around the aperture 18a. This can be achieved by making cuts to create slits 31 and 41 such that they are radially offset relative to each other. The maximum degree of offset will depend on the number of slits. In general, if slits are equally spaced, the maximum possible offset will be one half of the angle between the loops. For example, if distal side 30 (or proximal side 40) contains 4 slits (and therefore 4 loops), loops will be 90 degrees apart (see the formula described above), thereby allowing for maximum degree of offset of one half of 90 degrees (which is 45 degrees) between loops 32 and loops 42. In a preferred form, when distal side 30 (or proximal side 40) contains 4 slits (and therefore 4 loops), loops 42 and loops 32 are offset by 45 degrees. In an alternative embodiment, the degree of offset between loops 32 and 42 ranges from about 30 to about 45 degrees.

FIGS. 2E-2H illustrate another embodiment of the invention, where the occluder 20 is formed from a tube with loops 232 and 242, produced from the cutting pattern shown in FIG. 2E. In one embodiment, the proximal side 40 and the distal side 30 of occluder 20 each include eight loops or petals. As shown in FIG. 2E, the distal portion 30 of the tube 25 includes 8 slits 231 that form 8 extended segments of the tube that form the distal loops or petals 232. As apparent from the figures, the slits extend the entire distance of the distal portion 30 of the tube 25, i.e. between central tube 22 and distal end 39, so that the loops of identical cross-sections are formed. Upon application of force F_{d} to distal end 39, extended segments defined by slits 231 bow and twist outward to form distal petals 232 in distal side 30 of the occluder 20. The movement of the segments during deployment is such that the segments rotate in an orthogonal plane relative to the axis of the device. Central tube 22 may be constrained during the application of force F_{d}, or any combination of forces sufficient to reduce the axial length of the tube may be applied. One end of each of distal petals 232 originates from central tube 22, while the other end originates from distal end 39. Proximal petals 242 may be formed in proximal portion 40, as shown in FIGS. 2E-2H, making slits 241 between central tube 22 and proximal tip 44, using the same cutting pattern described above and applying force Fₚ or combination of forces sufficient to reduce the axial length of the tube by allowing slits 241 to bow and twist outward to form proximal petals 242 in proximal portion 40 of the occluder 20. One end of each of proximal petals 242 originates from central tube 22, while the other end originates from proximal tip 44.

As illustrated, the loops 232 are evenly distributed about central tube 22 and end 39. Thus, when proximal side 30 includes eight loops 232 (as shown in FIGS. 2G and 2H), the eight slits 231 are spaced 45 degrees radially apart. The angle between radially equally-spaced slits 231 in distal side 30 is determined by the formula (360/n_{d}) where n_{d} is the total number of loops 232.

The proximal side 40 of the occluder 20, shown in side view in FIG. 2H, also includes eight loops, 242a, 242b, 242c, 242d, 242e, 242f, 242g, and 242h (collectively referred to as loops 242). As previously described, each of loops 242a-242h is produced by cutting slits 241. The application of force Fₚ to tip 44 of tube 25 brings the axial ends of slits 241 together such that struts bow and twist outwardly to form loops 242 of proximal side 40 (FIGS. 2G-2H). Central tube 22 may be constrained during the application of force Fₚ. One skilled in the art will recognize that any combination of forces sufficient to reduce the axial length of the tube 25 would be sufficient to deploy the proximal side 40 of occluder 20. As described above for distal side 30, the loops 242 are evenly distributed about central tube 22 and tip 44. Similarly, the angle between radially equally-spaced slits 241 in proximal side 40 is determined by the formula (360/n_{d}) where n_{d} is the total number of loops 242.

Although the distal side 30 and the proximal side 40 of the occluder 20, shown in FIG. 2H, each include eight loops 232 and 242, respectively, one skilled in the art will recognize that the distal side 30 and proximal side 40 of an occluder 20 according to the present invention may include any number of loops 232 and 242, respectively, required and/suitable for a given application. Further, although as illustrated, distal side 30 and proximal side 40 both include eight loops, there is no requirement that distal side 30 and proximal side 40 include the same number of loops. In fact, in particular applications, it may be advantageous to use an occluder 20 in which distal side 30 contains fewer loops than proximal side 40, or vice versa.

It will be apparent to one skilled in the art that loops 232 and loops 242 do not have to be the same size. In one embodiment, loops 232 are larger in size than loops 242. In another embodiment, loops 232 are smaller in size than loops 242. Size of loops 232 and 242 is determined by the lengths of slits 231 and 241, respectively. Therefore, absolute and relative lengths of slits 231 and 241 can be varied to achieve desired absolute and relative sizes of loops 232 and 242.

While loops 232 and 242, shown in FIGS. 2F-2H are illustrated as aligned, this does not have to be the case. In one embodiment, loops 232 and 242 are radially offset from each other. This can be achieved by making cuts to create slits 231 and 241 such that they are radially offset relative to each other. The maximum degree of offset will depend on the number of slits. In general, if slits are equally spaced, the maximum possible offset will be one half of the angle between the loops. For example, if distal side 30 (or proximal side 40) contains 8 slits (and therefore 8 loops), the loops will be 45 degrees apart (see the formula described above), thereby allowing for maximum degree of offset of one half of 45 degrees, which is 22.5 degrees between loops 232 and loops 242. It is understood, that offset can be in either rotational direction (i.e., clockwise and counterclockwise). Therefore, in this example with 8 slits, an offset of 30 degrees is equivalent to an offset of 7.5 degrees in the opposite direction.

The cutting pattern illustrated in FIG. 2E can be varied, as shown in FIGS. 2I-2K. According to one embodiment of the invention, the number of slits 231 and 241 cut in the tube 25 can be changed according to the desired number of loops 232 and 242 in the occluder 20 when deployed. The cross-sectional dimensions of loops 232 and 242 are determined by the thickness of tube 25 and the distance between adjacent slits 231 and 241. The length of slits 231 and 241 determines the length of loops 232 and 242 and the radial dimensions of the deployed occluder 20. In this manner, the dimensions of loops 232 and 242 can be controlled during production of occluder 20. For example, as more material is removed from tube 25 during the cutting process used to form slits 231 and 241, the thickness of loops 232 and 242 decreases. Moreover, any or all of slits 231 and 241 can be cut such that thickness of loops 232 and 242 varies along their length. In some embodiments, it may be desirable to have wider loops 232 and 242 at the location where the loops join tube 25 to create a sturdier device. Alternatively, it may be desirable to have a wider portion elsewhere along the loops 232 and 242 such that occluder 20 is predisposed to bend into a certain shape and arrangement. For example, the portion of loops 232 and 242 nearer central tube 22 may be thinner than the portion of loops 232 and 242 nearer end 39 and tip 44, respectively, to facilitate bending of the loops 232 and 242.

Slits 231 and 241, as shown in FIG. 2J, are cut axially along the length of tube 25. However, as one of skill in the art will recognize, slits 231 and/or 241 may also be cut along other dimensions of tube 25. For example, as shown in FIG. 2I, slits 231 and 241 may be cut at an angle such that they are helically disposed on tube 25. Angled slits 231 and 241 produce angled loops 232 and 242 during deployment. Further, slits 231 and 241 need not be straight; for example, slits 231 and 241 may be cut as zigzags, S-shaped slits, or C-shaped slits. One skilled in the art will be capable of selecting the angle for the slits 231 and/or 241 and the loop 232 and 242 shape(s) appropriate for a given clinical application. For example, when occluder 20 is formed from a polymer tube 25, straight loops 232 and 242 may be preferable because they will impart maximum stiffness to occluder 20. If the tube 25 is formed of a stiffer material, the angled slits 231 and/or 241 may provide a more desired stiffness to the occluder 20.

In one embodiment, the occluder 20 has loops according to FIGS. 2A-2D on one side and loops according to FIGS. 2E-2H on the other side. For example, occluder 20 may comprise loops 42 on the proximal side 40 and loops 232 on the distal side 30, or it may comprise loops 242 on the proximal side 40 and loops 32 on the distal side 30.

In one embodiment, for example as shown in FIG. 2H, each loop 242 and 232 has some amount of twist, i.e., when the loop is formed, the proximal side of the loop is radially offset with respect to the distal side of the loop. Loops 242 and/or 232, however, need not have any twist.

FIG. 2M, for example, illustrates an embodiment of the occluder with slits cut as illustrated in FIG. 2L. In this embodiment, neither loops 32 nor loops 42 are twisted. It will be apparent to one skilled in the art that any combination of twisted and untwisted loops may be used. Furthermore, an occluder can have any combination of loops with different bends and twists if desired.

In one embodiment, loops 32 (or 232) of distal side 30 are bent to form concave loops, while loops 42 (or 242) of proximal side 40 are flat. In this embodiment, the outermost portions of loops 42 (or 242) of proximal side 40 oppose the outermost portions of the loops 32 (or 232) of the proximal side 30, as described in more detail below, thereby creating a desirable opposing force that secures the occluder 20 at its desired location in vivo. So configured, the opposing compressive forces exerted by sides 30 and 40 on the septum 12 following deployment of occluder 20 in vivo is advantageous in certain circumstances, such as closing certain kinds of PFOs. In another embodiment, loops 42 (or 242 of the proximal side 40 are bent, while loops 32 (or 232) of the distal side 30 are flat. In yet another embodiment, loops 42 (or 242) of the proximal side 40 and loops 32 (or 232) of the distal side 30 are bent.

Whatever the number and shapes of loops 32 and 42 (or 232 and 242), the loops 32 and 42 (or 232 and 242) may be of varied sizes to facilitate delivery of occluder 20, e.g. to improve collapsibility of the occluder 20 or to enhance its securement at the delivery site. For example, loops 32 and 42 (or 232 and 242) that are sized to better conform with anatomical landmarks enhance securement of the occluder 20 to the septum 12 in vivo. As indicated above, the cross-sectional dimensions of loops 32 and 42 (or 232 and 242) are determined by the thickness of tube 25 and the distance between adjacent slits 31 and 41 (or 231 and 241). The length of slits 31 and 41 (or 231 and 241) determines the size of loops 32 and 42 (or 232 and 242) and the radial extent of the deployed occluder 20. In at least some embodiments, each of distal side 30 and proximal side 40 has a diameter in the range of about 10 mm to about 45 mm, with the particular diameter determined by the size of the particular defect being treated. In particular embodiments, the diameter of distal side 30 will be different than that of proximal side 40 so as to better conform to the anatomy of the patient's heart.

The tube(s) 25 forming occluder 20 includes a biocompatible metal or polymer. In at least some embodiments, the occluder 20 is formed of a bioabsorbable polymer, or a shape memory polymer. In other embodiments, the occluder 20 is formed of a biocompatible metal, such as a shape memory alloy (e.g., nitinol). The thermal shape memory and/or superelastic properties of shape memory polymers and alloys permit the occluder 20 to resume and maintain its intended shape in vivo despite being distorted during the delivery process. In addition, shape memory polymers and metals can be advantageous so that the structure of the device assists in compressing the PFO tunnel closed. Alternatively, or additionally, the occluder 20 may be formed of a bioabsorbable metal, such as iron, magnesium, or combinations of these and similar materials. Exemplary bioabsorbable polymers include polyhydroxyalkanoate compositions, for example poly-4-hydroxybutyrate (P4HB) compositions, disclosed in U.S. Pat. No. 6,610,764, entitled Polyhydroxyalkanoate Compositions Having Controlled Degradation Rate and U.S. Pat. No. 6,548,569, entitled Medical Devices and Applications of Polyhydroxyalkanoate Polymers, both of which are incorporated herein by reference in their entirety.

The cross-sectional shape of tube 25 may be circular or polygonal, for example square, or hexagonal. The slits 31 and 41 (or 231 and 241) may be disposed on the face of the polygon (i.e., the flat part) or on the intersection of the faces. Various other cross-sectional shapes may also be used, examples of which are illustrated in FIGS. 2N-2R. The cross-sections of tube 25 may have different shapes at the inner and outer circumference. The outer portion of the tube 25 may have a ribbed surface in certain embodiments; the corresponding cross-section will be notched, or flower-like at the outer circumference depending on the shape and number of the ribs. The cross-section shown in FIG. 2N has an inner circumference 72 and an outer circumference 71. The inner circumference 72 is smooth and the outer circumference 71 is flower-like. The cross-section shown in FIG. 2P has a flower-like outer circumference 73 and a notched inner circumference 74. Both the outer and inner surfaces are ribbed. The cross-section shown in FIG. 2Q has a star-like outer circumference 75 and a smooth inner circumference 76. The cross-section shown in FIG. 2R has a squared-off flower-like outer circumference 77 and a smooth inner circumference 78. Using various cross-sectional geometrics (i.e., ribbing patterns) can improve implant performance by improving septal surface contact and opposition, mechanical performance, and/or ease of manufacturing. Cutting slits can be distributed anywhere evenly through the cross-sections, but preferably in the cavity portions of the outer surface of tube 25, between the ribs.

The tube 25 can be extruded or constructed of a sheet of material and rolled into a tube. The sheet of material could be a single ply sheet or multiple ply. The slits that form the struts could be cut or stamped into the tube prior to rolling the tube to connect the ends to form an enclosed cross section. Various geometrical cross sections are possible including circular, square, hexagonal and octagonal and the joint could be at the vertex or along the flat of a wall if the cross section is of a particular geometry. Various attachment techniques could be used to join the ends of the sheet to form a tube, including welding, heat adhesives, non-heat adhesives and other joining techniques suitable for in-vivo application.

The surface of tube 25 may be textured or smooth. An occluder 20 having a rough surface produces an inflammatory response upon contact with septum 12 in vivo, thereby promoting faster tissue ingrowth, healing, and closure of aperture 18a (shown in FIG. 1). Such a rough surface may be produced, for example, by shaving tube 25 to produce whiskers along its surface. For example, central tube 22 may include such whiskers. Additionally or alternatively, the surface of tube 25 may be porous to facilitate cell ingrowth.

As indicated previously and shown in FIGS. 2A-2H, distal side 30 and proximal side 40 of occluder 20 are connected by central tube 22. The central tube 22 is formed by the portion of tube 25 between the distal side 30 of tube 25, which contains slits 31, (or 231) and the proximal side 40 of tube 25, which contains slits 41 (or 241). Given that the central portion of tube 25 remains uncut during the cutting process, the central portion of the tube maintains its profile upon the application of forces F_{d} and Fₚ and does not bow and twist outward as the proximal and distal sides are adapted to do.

According to one embodiment, central tube 22 is straight, as illustrated in FIGS. 2D and 2H, where the central tube 22 is perpendicular to loops 32 and 42 (or 232 and 242). According to another embodiment of the invention, central tube 22 is positioned at an angle θ relative to the proximal side 40 of the occluder 20, as shown, for example, in FIG. 5B. The shape of central tube 22 included in a given occluder is, at least in part, determined by the nature of the aperture 18. An occluder having a straight central tube 22 is particularly suited to treat an anatomical anomaly including a perpendicular aperture, such as an ASD and certain PFOs. Often, however, anatomical anomalies, such as certain PFOs, have non-perpendicular apertures and are sometimes quite significantly non-perpendicular. An occluder having an angled central tube 22 is well-suited for treatment of such defects, such that the angle of the anatomical aperture 18 is more closely matched by the pre-formed angle θ of the occluder 20. Also, the length of central tube 22 can be varied depending on the anatomy of the defect being closed. Accordingly, the distal side 30 and proximal side 40 of occluder 20 are more likely to be seated against and minimize distortion to the septum 12 surrounding the aperture 18, as shown in FIG. 8. A well-seated occluder 20 is less likely to permit blood leakage between the right 11 and left 13 atria, and the patient into which the occluder 20 has been placed is, therefore, less likely to suffer embolisms and other adverse events.

Advantageously, angled central tube 22 also facilitates delivery of occluder 20 because it is angled toward the end of the delivery sheath. In at least some embodiments, the angle θ is about 0-45 degrees. To form the angle θ, proximal side 40 of the occluder 20 bends depending upon, among other factors, the material used to form occluder 20. Accordingly, depending upon design considerations, tip 44 and end 39 may be aligned with central tube 22 or perpendicular to proximal side 40 or some variation in between. One skilled in the art will be capable of determining whether a straight or angled central tube 22 is best suited for treatment of a given anatomical aperture 18 and the appropriate angle θ, typically in the range between about 30 and about 90 degrees. Sometimes, angles of about 0 degrees to about 30 degrees can be used in an oblique passageway such as a very long tunnel PFO. One skilled in the art will recognize that the concept of an angled central tube may be applied to septal occluders other than those disclosed herein.

When central tube 22 is positioned at angle θ, distal side 30 and proximal side 40 of occluder 20 may be configured such that they are either directly opposing or, as shown in FIG. 5B, offset by distance A. One skilled in the art will, of course, recognize that the shape and arrangement of either or both of distal side 30 and proximal side 40 may be adjusted such that the compressive forces they apply are as directly opposing as possible. However, in some clinical applications, an occluder 20 having an offset of distance A may be particularly desirable. For example, as shown in FIG. 5B, if the septum 12 surrounding aperture 18 includes a disproportionately thick portion (e.g. septum secundum 16 as compared to septum primum 14), the offset A may be used to seat occluder 20 more securely upon septum 12. Moreover, the offset A allows each of sides 30 and 40 to be centered around each side of an asymmetric aperture 18.

When occluder 20 is delivered in vivo, a marker is required to properly orient the occluder 20 in its intended delivery location. For example, a platinum wire may be wrapped around one of loops 32 or 42 (or one of loops 232 or 242) so as to permit visualization of the orientation of the occluder 20 using fluoroscopy. Alternatively, other types of markers may be used, e.g. coatings, clips, etc. As one skilled in the art would appreciate, the radiopaque marker could be blended in with the extrudate and thus provide visibility under fluoroscopy. As will be readily understood by one skilled in the art, the orientation of a non-symmetrical occluder 20 during delivery is of great importance.

Upon deployment in vivo (a process described in detail below), an occluder 20 according to the present invention applies a compressive force to the septum 12. Distal side 30 is seated against the septum 12 in the left atrium 13, central tube 22 extends through the aperture 18, and proximal side 40 is seated against the septum 12 in the right atrium 11. At least some portion of each of loops 32 and 42 (or 232 and 242) contacts septum 12. In particular embodiments, a substantial length of each of loops 32 and 42 (or 232 and 242) contacts septum 12. As illustrated in the representative Figures, the proximal side 40 and distal side 30 of occluder 20 overlap significantly, such that the septum 12 is “sandwiched” between them once the occluder 20 is deployed. According to at least some embodiments and depending upon the material used to form occluder 20, the loops 32 and 42 (or 232 and 242) provide both a radially-extending compressive force and a circumferential compressive force to septum 12. In these embodiments, the compressive forces are more evenly and more widely distributed across the surface of the septum 12 surrounding the aperture 18 and, therefore, provide the occluder 20 with superior dislodgement resistance as compared to prior art devices. As used in this application, “dislodgement resistance” refers to the ability of an occluder 20 to resist the tendency of the force applied by the unequal pressures between the right 11 and left 13 atria (i.e. the “dislodging force”) to separate the occluder 20 from the septum 12. Generally, a high dislodgement resistance is desirable.

Loops 32 and 42 (or 232 and 242) are also configured to minimize the trauma they inflict on the septum 12 surrounding aperture 18. Specifically, as indicated previously, the outer perimeter of loops 32 and 42 (or 232 and 242) may be rounded.

According to one embodiment of the invention, for example, as illustrated in FIGS. 2B-2D, the circumferential portions of loops 32 and 42 are thinner than the orthogonally-extending portions of loops 32 and 42; therefore, the center of the occluder 20 is stronger than its perimeter. Accordingly, outer perimeter of loops 32 and 42 of occluder 20 has a low compression resistance. As used in this application, “compression resistance” refers to the ability of an occluder 20 to resist the lateral compressive force applied by the heart as it contracts during a heartbeat. Generally, an occluder that resists compressive force, i.e. has high compression resistance, is undesirable because its rigid shape and arrangement may cause trauma to the septum 12, the right atrium 11, and/or the left atrium 13.

According to at least some embodiments of the present invention, occluder 20 further includes a catch system, generally indicated at 131, that secures the occluder 20 in its deployed state. The catch system 131, in general, maintains the shape and arrangement of loops 32 and 42 (or 232 and 242) of occluder 20, once the occluder 20 has been deployed. Catch system 131 reduces and maintains the axial length of the occluder 20 so that occluder 20 maintains its deployed state, is secured in the aperture 18, and consistently applies a compressive force to septum 12 that is sufficient to close aperture 18. Catch system 131 is particularly advantageous when the occluder 20 is formed of a polymeric material, as previously described, because the polymeric occluder 20 may be deformed during delivery such that it may not fully recover its intended shape once deployed. By reducing and maintaining the axial length of occluder 20 once it has been deployed in vivo, catch system 131 compensates for any undesirable structural changes suffered by occluder 20 during delivery. In some embodiments, catch system 131 includes a ceramic material or a material selected from the group consisting of metals, shape memory materials, alloys, polymers, bioabsorbable polymers, and combinations thereof. In particular embodiments, the catch system may include nitinol or a shape memory polymer. Further, the catch system may include a material selected from the group consisting Teflon-based materials, polyurethanes, metals, polyvinyl alcohol (PVA), extracellular matrix (ECM) or other bioengineered materials, synthetic bioabsorbable polymeric scaffolds, collagen, and combinations thereof.

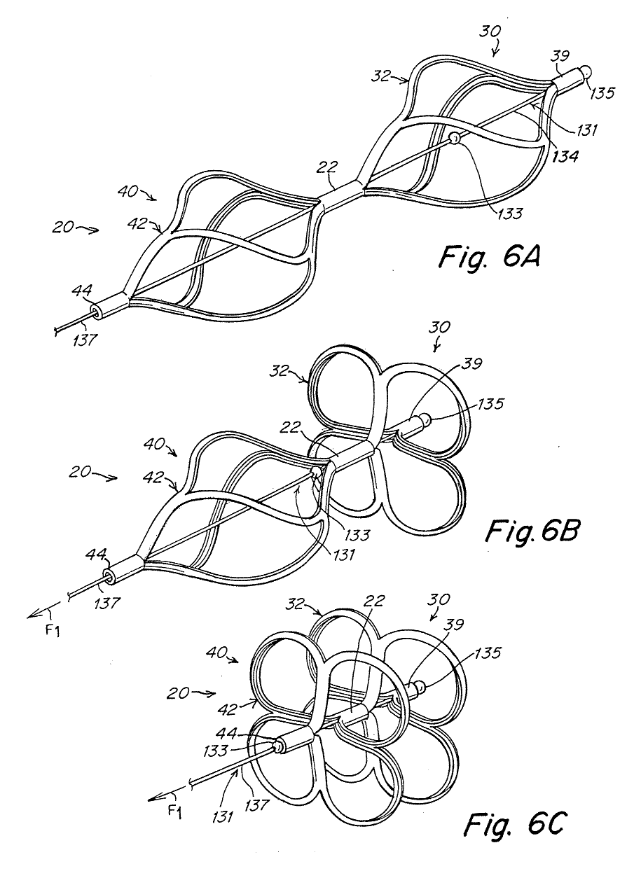

Catch system 131 may take a variety of forms, non-limiting examples of which are provided in FIGS. 6A-6C. For example, as shown in FIG. 6A, catch system 131 includes two catch elements, e.g., balls, 133 and 135, connected by wire 134. The catch system and catch element are preferably made of the same material as the occluder, although based on design selection, they could be made of the same or different material. In certain circumstances, it may be necessary to make them of different material. As illustrated in FIG. 6A, delivery string 137 is attached to ball 133 and is then extended through end 39, distal portion 30 of tube 25, central tube 22, proximal portion 40 of tube 25, and tip 44, such that ball 133 is located between central tube 22 and end 39 and ball 135 is located on the distal side of central tube 22. The function of catch system 131 is shown in FIGS. 6B-6C. Ball 133 is designed such that, upon the application of sufficient pulling force F₁ to delivery string 137, it passes through central tube 22 (FIG. 6B) and tip 44 (FIG. 6C). Ball 133 cannot reenter tip 44 or central tube 22 without the application of a sufficient, additional force. In this manner, ball 133 may be used to bring together the distal side 30 and the proximal side 40, thereby reducing and maintaining the axial length of occluder 20. Obviously, during the application of pulling force F₁, the tip 44 of occluder 20 must be held against an object, such as a delivery sheath. Ball 135 is designed such that, upon application of sufficient pulling force F₂ to delivery string 137, it passes through end 39 and central tube 22. The pulling force F₂ required to move ball 135 through end 39 and central tube 22 is greater than the pulling force F₁ required to move ball 133 through central tube 22 and tip 44. However, ball 135 cannot pass through tip 44. Thus, the application of sufficient pulling force F₂ to ball 135 releases distal side 30 and proximal side 40, as described in more detail below. It should be noted that while catch elements 133 and 135 are illustrated as spherical elements in FIGS. 6A-6C, catch elements 133 and 135 may take any suitable shape. For example, catch elements 133 and 135 may be conical. The narrow portions of conical catch elements 133 and 135 point toward tip 44 of proximal side 40. One possible mode of recovery or retrieval for this device is simply reversing the implantation procedure. Of course, other modes of recovery or retrieval are possible, some of which are described in this specification.

Occluder 20 may be prepared for delivery to an aperture 18 in any one of several ways. Slits 31 and 41 (or 231 and 241) may be cut such that tube 25 bends into its intended configuration following deployment in vivo. Specifically, slits 31 and 41 (or 231 and 241) may be cut to a thickness that facilitates the bending and formation of loops 32 and 42 (or 232 and 242). Upon the application of forces F_{d} and Fₚ, tube 25 bends into its intended deployed configuration. Alternatively and/or additionally, tube 25 formed of a shape memory material may be preformed into its intended configuration ex vivo so that it will recover its preformed shape once deployed in vivo. According to at least some embodiments, these preforming techniques produce reliable deployment and bending of occluder 20 in vivo. An intermediate approach may also be used: tube 25 may be only slightly preformed ex vivo such that it is predisposed to bend into its intended deployed configuration in vivo upon application of forces F_{d} and Fₚ.

An occluder 20 as described herein may be delivered to an anatomical aperture 18 using any suitable delivery technique. For example, distal side 30 and proximal side 40 of occluder 20 may be deployed in separate steps, or both distal side 30 and proximal side 40 of occluder 20 may be deployed in the same step.

As shown in FIGS. 7A-7B, a delivery sheath 161 is used to deliver occluder 20 including the catch system 131 illustrated in FIGS. 6A-6C. Sheath 161 contains occluder 20 in its elongated, delivery form (FIG. 7A). As shown in FIG. 7B, delivery sheath 161 is first inserted into the right atrium 11 of the patient's heart. The full deployment and delivery sequence according to one embodiment is detailed in U.S. application Ser. No. 11/395,718, which has the same assignee as the present application and is incorporated herein in its entirety by reference. When properly deployed, occluder 20 rests within the aperture 18, and the distal side 30 and proximal side 40 exert a compressive force against septum primum 14 and septum secundum 16 in the left 13 and right 11 atria, respectively, to close the aperture 18, i.e. the PFO.

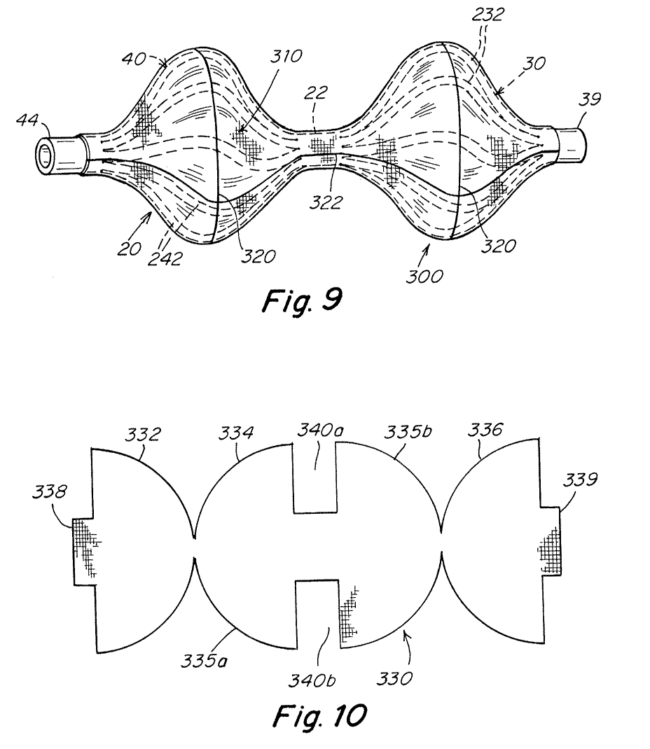

Occluder 20 may be modified in various ways. According to some embodiments of the present invention, distal side 30 and/or proximal 40 side of occluder 20 may include a tissue scaffold. The tissue scaffold encases the whole or part of the occluder 20 or extends across openings between the loops 232, 242 of the occluder. The tissue scaffold ensures more complete coverage of aperture 18 and promotes encapsulation and endothelialization of septum 12, thereby further encouraging anatomical closure of the septum 12. Various embodiments of tissue scaffolds for use with a tubular occluder are shown in FIGS. 9 through 14A-C. The tissue scaffold is described and illustrated with respect to the occluder of FIGS. 2E-H; however, a tissue scaffold as discussed in the present application may be used with any tubular occluder, including other embodiments described herein and those described in U.S. application Ser. No. 11/395,718 or U.S. application Ser. No. 10/890,784, both of which applications have the same assignee as the present application and are incorporated herein in their entireties by reference.

As shown in FIG. 9, a scaffolded occluder 300 includes an occluder 20 and a tissue scaffold 310. In this embodiment, the tissue scaffold 310 completely encapsulates the occluder petals 232, 242, and the center tube 22. The coverage provided by tissue scaffold 310 offers several advantages, including that the tissue scaffold 310 improves the sealing of the aperture being closed. Another advantage is that the tissue scaffold can enhance the implant's stability at the desired delivery location. The tissue scaffold can allow and facilitate the ingrowth of tissue, certain pharmacological agents can be applied or embedded in the tissue scaffold for delivery to the implant site.

The tissue scaffold 310 is formed and attached to the occluder frame 20 in a series of steps. FIG. 10 illustrates a tissue scaffold component 330. Two tissue scaffold components 330 are used to form a single tissue scaffold 310. Tissue scaffold component 330 may be cut as multiple pieces and then joined together into the illustrated shape, but is preferably formed of a single piece of tissue scaffold material. Suitable materials are discussed herein below but will generally be any biocompatible, flexible material with the desired thickness, flexibility and other properties for the application. Tissue scaffold component 330 generally includes a first semicircular end flap 332, a central element 334 incorporating two semicircular middle flaps 335a and 335b, and a second semicircular end flap 336. The flaps are designed to fold around the loops of the occluder 20 so that when joined together, two tissue scaffold components 330 approximate the shape of and wrap around the occluder 20. Semicircular end flap 332 includes a rectangular tab 338. Semicircular end flap 336 includes a rectangular tab 339. The central element 334 includes two rectangular cutouts 340a and 340b. The rectangular tabs 338 and 339 and the two rectangular cutouts 340a and 340b are designed to accommodate the proximal end 44, the distal end 39, and the center tube 22 of the occluder frame.

To assemble the tissue scaffold 310, one tissue scaffold component 330 is lined up with a second tissue scaffold component 330. All of the matching straight edges, except for the far left edge on tab 338 and the far right edge on tab 339, are sealed to form a generally tubular profile. The connected curved edges of each scaffold component 330 are then joined to the corresponding edges of the other component. The tissue scaffold 310 thus formed is then turned inside out. This ensures that the seams formed in the assembly process are on the inside of the tissue scaffold 310. The resulting tissue scaffold 310 has an inside pocket and a center opening that extends from one end to the other, with the far left edge and the far right edge left open. An elongated occluder 20 is then inserted through the center opening of the tissue scaffold pocket. After aligning the proximal 44 and distal ends 39 with the respective ends of the tissue scaffold, the occluder is relaxed, and its position is adjusted within the pocket. When the proper placement is achieved, the proximal end of the tissue scaffold 310 is sealed to the proximal end of the occluder, and the distal end of the tissue scaffold 310 is sealed to the distal end of the occluder. As illustrated in FIG. 9, tissue scaffold 310 includes seams, such as seams 320 and 322. The presence of such seams may impact the dimensions of the occluder, the size of catheter to be used and other aspects of the use of the occluder.

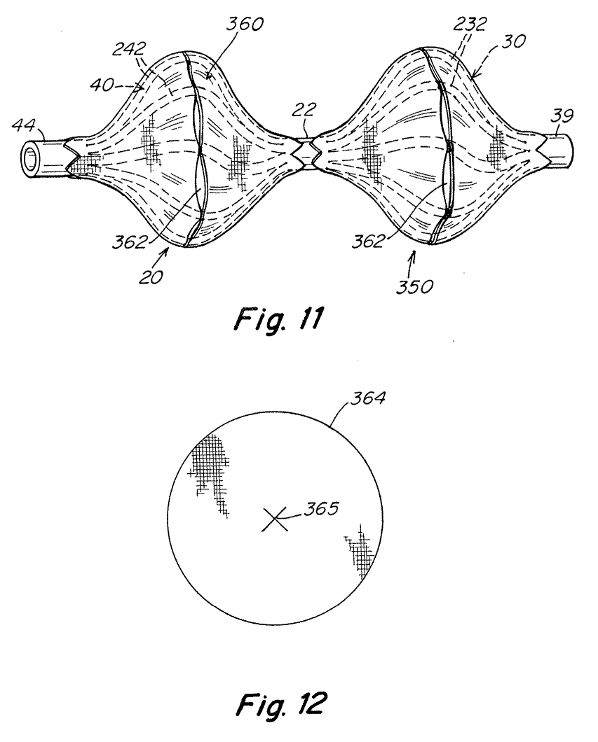

FIG. 11 illustrates another embodiment 350 of a tubular occluder with a tissue scaffold. The tissue scaffold 360 has fewer seams (and reduced seam length) relative to tissue scaffold 310. The proximal 40 and distal sides 30 of the proximal and distal petals 42 and 32 are covered with tissue scaffold 360. The petals 242 and 232 are not completely encapsulated by the tissue scaffold 360. Although illustrated as petals, the tissue scaffold of the present invention may be used with a variety of occluder configurations. In those circumstances, the multiple pedals formed by occluder as it is deployed can be termed anchoring portions. That is, there is a proximal anchoring portion and a distal anchoring portion that cooperate on each side of the defect or physical anomaly (e.g., PFO) so that the device is fixed in place once deployed. Small openings 362 in the tissue scaffold 360 are present around the outer edges of the proximal and distal petals 242 and 232.

The tissue scaffold 360 is composed of four disks of scaffold material, such as the disk 364 illustrated in FIG. 12. The disk 364 includes a center slit 365 in the form of an X, or other aperture. The center slit 365 enables the elongated occluder to be inserted through the disks so that the disks slide over the tube into their respective positions. One disk is then bonded to each of the proximal and distal (seen in the figures as left and right) sides of the proximal and distal petals 242 and 232. The outer edges of the disks are not bonded to each other, leaving openings 362. This design eliminates an edge seam (previously identified by reference numeral 320) and may reduce the overall profile of the wrapped occluder. In an alternative embodiment, the outer edges of the disks may be bonded to each other only at a few discrete points. A rectangular piece can also be wrapped around the center tube 22 and bonded to it, if coverage of the center tube 22 is desired. Alternatively, a preformed tube can also be slid down the elongated occluder to cover the center tube 22. In addition, the distal and proximal ends 39 and 44 can also be covered by tissue scaffold material, for example, by sliding a preformed tube over the ends or by bonding a precut flat piece of tissue scaffold to the desired portion of the occluder.

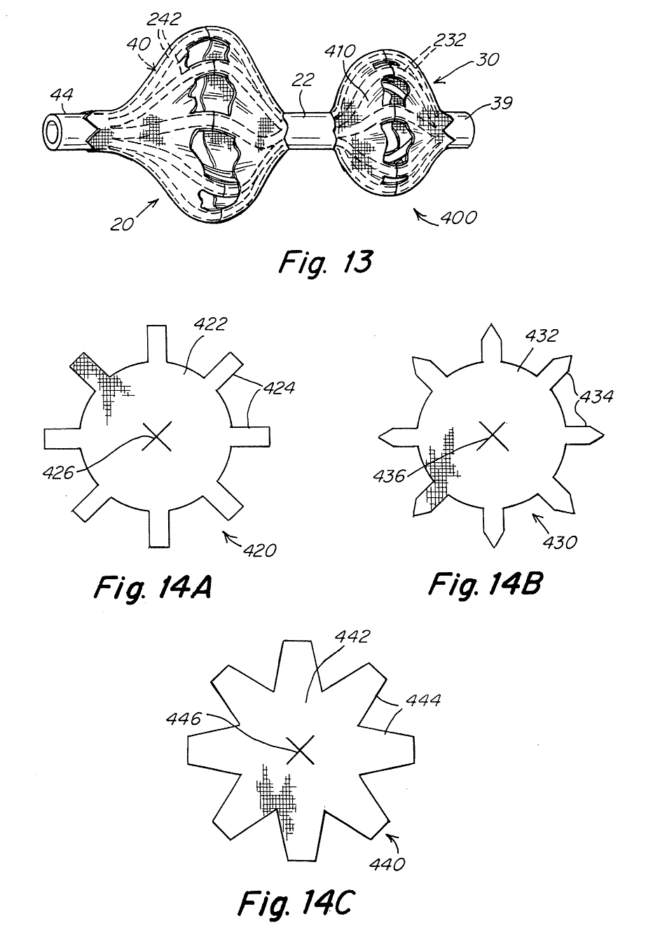

FIG. 13 illustrates yet another embodiment 400 of a tubular occluder with a tissue scaffold. Similar to tissue scaffold 360, tissue scaffold 410 covers the sides of the proximal and distal petals 42 and 32 with separate pieces of tissue scaffold material. As shown in FIGS. 14A-C, the scaffold pieces 420, 430, and 440 are adapted to the shape of the proximal and distal petals 42 and 32 and have a “sunflower” shape. The scaffold pieces 420, 430, and 440 have a solid center portion 422, 432, and 442, with a generally circular shape, and radially extending projections 424, 434, and 444 of different shapes. A center slit 426, 436, and 446 is also provided. To make a tissue scaffold, four pieces such as scaffold pieces 420 are bonded at their centers 422 to the proximal and distal sides of the proximal and distal petals 42 and 32. The flaps formed by radially extending projections 424 may be individually bonded to individual struts of proximal and distal petals 42 and 32. The number, shape and dimensions of the radially extending projections, such as projections 422, will depend on the particular tubular occluder and the application. This embodiment 400 further reduces the edge profile of the occluder. This permits the use of a smaller delivery sheath, which is generally more desirable. If coverage is desired, the center tube 22 can be covered by bonding a separate rectangular piece of scaffold material to the center tube 22. Alternatively, a preformed tube can also be slid down the elongated occluder to cover the center tube 22. In addition, the distal and proximal ends 39 and 44 can also be covered by tissue scaffold material, for example, by sliding a preformed tube over the ends or by bonding a precut flat piece of tissue scaffold to the desired portion of the occluder.

The tissue scaffold may be formed of any flexible, biocompatible material capable of promoting tissue growth, including but not limited to polyester fabrics, Teflon-based materials, ePTFE, polyurethanes, metallic materials, polyvinyl alcohol (PVA), extracellular matrix (ECM) or other bioengineered materials, synthetic bioabsorbable polymeric scaffolds, other natural materials (e.g. collagen), or combinations of the foregoing materials. For example, the tissue scaffold may be formed of a thin metallic film or foil, e.g. a nitinol film or foil, as described in United States Patent Publ. No. 2003/0059640 (the entirety of which is incorporated herein by reference). Also, the surface of the tissue scaffold can be modified with drugs or biological agents to improve the defect healing and/or to prevent blood clotting or for other therapeutic purposes. Loops 32 and 42, (or 232 and 242), can be laser welded, ultrasonically welded, thermally welded, glued, or stitched to the tissue scaffold to securely fasten the scaffold to occluder 20.

The size and shape of the tissue scaffold are adapted to fit the size and shape of the corresponding implant. A larger implant requires a larger tissue scaffold and a smaller implant requires a smaller tissue scaffold. In addition, an implant with a greater proximal profile and a smaller distal profile requires a tissue scaffold with a greater proximal profile and a smaller distal profile.

In those embodiments where occluder 20 includes a tissue scaffold, the scaffold may be located on the outside surface of distal side 30 and proximal side 40 of the occluder only, with an alternative embodiment of additionally including scaffold on the inside surface of distal side 30 and proximal side 40 of the occluder. Also, the tissue scaffold could be disposed against the tissue that is sought to be occluded, such as the septum 12 so that the proximity of the tissue scaffold and septal tissue 12 promotes endothelialization. One skilled in the art will be able to determine those clinical applications in which the use of tissue scaffolds and/or stitches is appropriate. When an occluder 20 with a tissue scaffold is elongated into its delivery configuration, the tissue scaffold is sufficiently flexible that it allows the occluder 20 to fold into its reduced profile configuration.

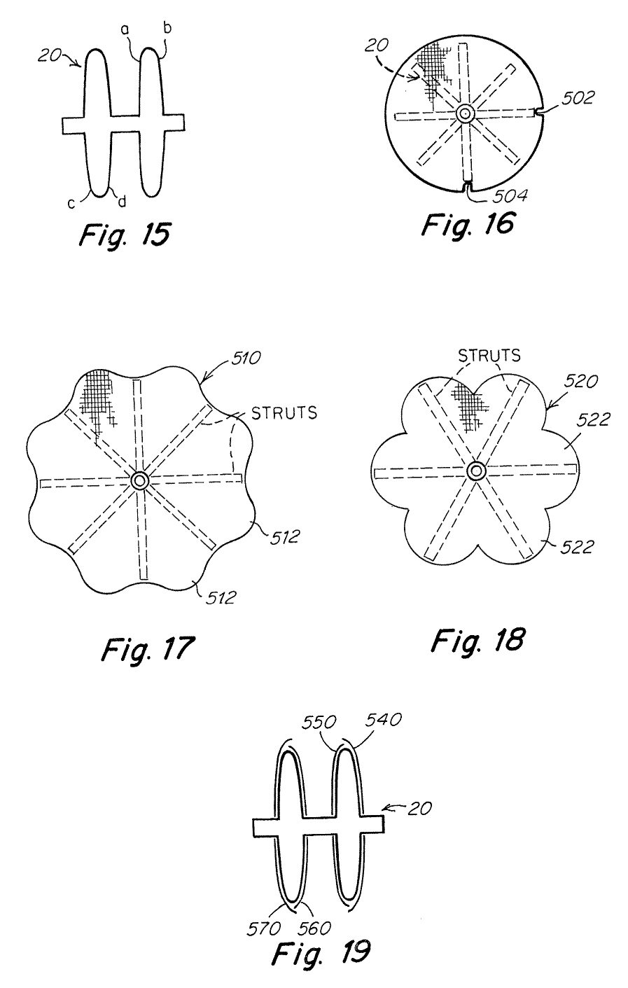

The preparation and attachment of the discs to the occluder will be described in connection with FIG. 15 which illustrates an occluder 20 in schematic form. A disc is cut with cutters to an appropriate size for the implant. As described earlier, rectangular shaped pieces can also be cut to cover the tubular portions of the occluder. In a presently preferred embodiment, a tubular piece of tissue scaffolding material is prepared to cover the distal, proximal ends and center tube. The relevant areas of the occluder for attachment are: a) proximal abluminal, b) proximal luminal, c) distal luminal, and d) distal abluminal. In general, the abluminal side of the anchor portion is the side nearest the tissue and the luminal side is the side furthest away. As noted above, four discs may be attached to the occluder to provide tissue scaffolding. The steps below can be used for any of the 4 discs that could be applied, e.g., the proximal abluminal, the proximal luminal, the distal abluminal and the distal luminal.

First the implant is placed on a suitable workstation, e.g., a mandrel. Next a disc is placed at the desired attachment location, e.g., the proximal abluminal location, and the center of the disc is melted onto the occluder struts. That is, the disc is affixed to the strut by the melted disc and struts. The disc may be melted by any number of melting techniques known in the art, including, but not limited to, direct heating, radiant heating, laser heating, and ultrasonic welding. In an embodiment of the invention, the disc is only affixed to the tips of each strut and not beyond, such as locations 502, 504 in FIG. 16. In an alternative embodiment, the disc is affixed along the struts of the occluder.

In a presently preferred embodiment, each strut is covered with scaffolding material. This process can be repeated for each of the discs that are used on the occluder. In a presently preferred embodiment, the luminal disc is attached first, then the loop tips and then the abluminal disc. It may require placing abluminal discs onto the occluder first so that they can be positioned prior to affixing the luminal sides. In one embodiment of the invention, the proximal and distal sides of the occluder are covered with 4 pieces of the scaffold material, i.e. 4 discs. In this embodiment with 4 discs, three of the discs have a center slit, while the fourth slit on the distal side of the occluder does not have a slit, and therefore completely covers the distal tip In an alternative embodiment, the proximal and distal sides of the occluder can be covered with more or less pieces of the scaffold material depending on the conveniences of the manufacturing process and other factors. For example, each occluder strut can be covered by an individual rectangular piece of scaffold material.

Alternate shapes can be used for the discs. For example, with reference to FIGS. 17 and 18, a disc 510 and 520, respectively, can be used with an occluder of the present invention. In this embodiment the outer edges of the discs can include scalloped shapes 512 and 522, respectively. The scallops can be aligned with the struts, as illustrated in FIG. 18, or the scallops can be off-set so that they are between the struts, as illustrated in FIG. 17. If the scallops are off-set from the struts, they can be folded over and affixed to an adjacent disc.

Finally, with reference to FIG. 19, the discs can be sized in a manner that can optimize the benefit of the tissue scaffold while not allowing the tissue scaffold to increase the delivery diameter beyond an acceptable amount. FIG. 19 illustrates a schematic occluder 20 with 4 discs that are in place to be scaffolding. The discs are not affixed to illustrate the relative size difference between the discs. Using the nomenclature introduced in FIG. 15, a disc 540 is disposed at the proximal luminal location, disc 550 is disposed at the proximal abluminal location, disc 560 is disposed at the distal abluminal section and disc 570 is disposed at the distal luminal section. In the presently preferred embodiment, the discs 540 and 570 are larger than the deployed diameter of the occluder and the discs 550 and 560 are the same size as the deployed diameter of the occluder. In the presently preferred embodiment, the discs 540 and 570 are 110% the diameter of the other discs and the deployed diameter of the occluder.

One skilled in the art will recognize that the occluders described herein may be used with anti-thrombogenic compounds, including but not limited to heparin and peptides, to reduce thrombogenicity of the occluder and/or to enhance the healing response of the septum 12 following deployment of the occluder in vivo. Similarly, the occluders described herein may be used to deliver other drugs or pharmaceutical agents (e.g. growth factors, peptides). The anti-thrombogenic compounds, drugs, and/or pharmaceutical agents may be included in the occluders of the present invention in several ways, including by incorporation into the tissue scaffold, as previously described, or as a coating, e.g. a polymeric coating, on the tube(s) 25 forming the distal side 30 and proximal side 40 of the occluder 20. Furthermore, the occluders described herein may include cells that have been seeded within the tissue scaffold or coated upon the tube(s) 25 forming the distal side 30 and proximal side 40 of the occluder 20.

One skilled in the art will further recognize that occluders according to this invention could be used to occlude other vascular and non-vascular openings. For example, the device could be inserted into a left atrial appendage or other tunnels or tubular openings within the body.

Having described preferred embodiments of the invention, it should be apparent that various modifications may be made without departing from the spirit and scope of the disclosure.

What is claimed is:

1. An occluder, comprising:
a first anchor member for deployment proximate a first end of a septal defect;
a second anchor member for deployment proximate a second end of said septal defect wherein at least one of the first and second anchor members includes radially expandable struts;
a connecting member connecting said first and second anchor members; and
a tissue scaffold attached to at least one of the radially expandable struts of the anchor members that improves the sealing between the occluder and the defect.

2. The occluder of claim 1, wherein said first and second anchor members and said connecting member comprise bioresorbable materials.

3. The occluder of claim 1 wherein a tissue scaffold is disposed on each of the first and second anchor members.

4. The occluder of claim 3 wherein a side of each of the first and second anchor members includes a tissue scaffold.

5. The occluder of claim 4 wherein a disc is attached to each side of the each of the first and second anchor members.

6. The occluder of claim 1 wherein the tissue scaffold is attached to an outer surface of the connecting member.

7. The occluder of claim 1 wherein the tissue scaffold includes a disc shaped material comprising an outer edge, wherein the outer edge of the disc shaped material is non-circular.

8. A septal defect closure device for closing a defect in heart tissue, comprising:
a first anchor member having a generally cylindrical tubular shape for deployment proximate a first end of a septal defect and an expanded shape for apposition against tissue;
a second anchor member having a generally cylindrical tubular shape for deployment proximate a second end of said septal defect and an expanded shape for apposition against tissue;
a connector member joining the first anchor member and second anchor member; and
a tissue scaffolding surrounding a substantial portion of the occluder.

9. The device of claim 8 wherein the tissue scaffolding includes a pharmacological agent for producing a desired physiological result.

10. The device of claim 8 wherein the tissue scaffolding includes at least two discs used to cover one of the first and second anchor members, wherein one disc is sized larger than the other.

11. The device of claim 10 wherein said one disc has a diameter that is 110% larger than the smaller disc.

12. The device of claim 11 wherein each disc comprises an outer edge, wherein the outer edge of one disc is folded over the outer edge of the other disc.

13. A septal defect closure device having a generally tubular delivery configuration and an expanded diameter deployed configuration, comprising:
an elongated proximal anchor member for deployment proximate a first end of a septal defect;
an elongated distal anchor member for deployment proximate a second end of said septal defect;
a connector member joining the proximal and distal anchor members; and,
a tissue scaffold attached to the septal defect closure device and configured to be disposed between the septal defect and the septal defect closure device.

14. The device of claim 13 wherein the tissue scaffold comprises a thrombogenic or inflammatory material.

15. The device of claim 14 wherein the tissue scaffold further comprises a bioabsorbable material.

16. The device of claim 14 wherein the tissue scaffold further comprises a biological material.

17. The device of claim 16 wherein the biological material comprises a purified bioengineered type I collagen.

18. The occluder of claim 17, wherein the purified bioengineered type I collagen is derived from a tunica submucosa layer of a porcine small intestine.

19. The device of claim 14 wherein said tissue scaffold is covered with a growth factor to accelerate tissue ingrowth.

20. A septal defect occluder, comprising:
a proximal anchor member for deployment proximate a first end of a septal defect;
a distal anchor member for deployment proximate a second end of said septal defect;
a flexible connector member connecting said proximal and distal anchor members; and
a tissue scaffolding formed from a plurality of discs affixed to at least one of the proximal and distal anchor members.

21. The occluder of claim 20 wherein said proximal and distal anchor members and said connection member comprise bioresorbable materials.

22. The occluder of claim 21 wherein a tissue scaffold is disposed on each of the proximal and distal anchor members.

23. The occluder of claim 21 wherein a side of each anchor member for contacting a tissue surface includes a tissue scaffold.

24. The occluder of claim 1 wherein tissue scaffolding is attached to the outer surface of the connecting member.

25. The method of attaching tissue scaffold to an occluder including the steps of:
a) cutting a first piece of scaffold material,
b) placing the first piece of scaffold material at the desired attachment location
c) melting the first piece of scaffold to the occluder,
d) cutting a second piece of scaffold material,
e) placing the second piece of scaffold material proximate to the first piece of scaffold material, and
f) melting the edge of the first piece of scaffold material to the edge of the second piece of scaffold material.
